(19) United States
(12) Patent Application Publication
(10) Pub. No.: US 2018/0317892 A1
(43) Pub. Date: Nov. 8, 2018

(54) STOOL COLLECTION, CONTAINMENT, AND SPECIMEN DISBURSEMENT SYSTEM

(71) Applicant: George CATLIN, Austin, TX (US)

(72) Inventor: George CATLIN, Austin, TX (US)

(21) Appl. No.: 16/030,217

(22) Filed: Jul. 9, 2018

Related U.S. Application Data
(63) Continuation-in-part of application No. 15/969,179, filed on May 2, 2018.
Application No. 15/969,179 is a continuation of application No. 14/874,423, filed on Oct. 3, 2015, now Pat. No. 9,986,978.

(51) Int. Cl. A61B 10/00; A47K 13/16; A47K 11/02

(52) U.S. Cl. CPC A61B 10/0038; A47K 11/02; A47K 13/16

(57) ABSTRACT

The invention relates to devices for collection, containment, storage and distribution of human stool samples for use in diagnostics related to infectious disease, IBD (Inflammatory Bowel Disease), IBS (Inflammatory Bowel Syndrome) and other disease states or clinical conditions.

### CROSS REFERENCE TO RELATED APPLICATIONS

This CIP patent application claims priority to pending U.S. patent application Ser. No. 15/969,179, which in turn claims priority to U.S. patent application Ser. No. 14/874,423 that issued as U.S. Pat. No. 9,986,978.

### FIELD OF THE INVENTION

The invention relates to devices for collection, containment, storage and distribution of human stool samples for use in diagnostics related to infectious disease, IBD (Inflammatory Bowel Disease), IBS (Inflammatory Bowel Syndrome) and other disease states or clinical conditions.

### BACKGROUND

Stool sample collection and subsequent handling for patients as ordered by physicians for diagnostics can be a tedious, messy and highly unpleasant process for patients. The vast amount of stool sample collections by patients are completed in the home and subsequently processed in a lab or diagnostic facility. Perhaps the most unpleasant aspect for patients is the handling and distribution (alternatively “disbursement”) required to transfer smaller stool samples into separate mediums or storage containers. The patient handling and/or disbursement can also lead to sample contamination and subsequent inaccurate diagnostic results. Further, the handling and distribution of feces by the patient or lab technicians can expose them to unsanitary or infectious agents within the stool sample itself. As a result of these unpleasant aspects, many patients decide not to submit their stool samples in compliance with doctor's orders.

After their clinician has ordered a stool sample, the patient is provided with a stool sample a collection device, supplied by the diagnostic center or by their doctor's office. Other ancillary materials are also supplied to the patient such as surgical gloves, tongue depressors (or wooden sticks), and a variety of containers or test tubes designated for specific tests or diagnostic processes.

Typically, the patient sits atop a collection device fastened to their toilet or the patient situates himself or herself above a paper drape or collection cup designed to catch the sample and prevent it from falling into the toilet bowl.

After the patient has defecated into the collection cup or drape, he or she is required to wear plastic gloves and use a tongue depressor or a small, plastic spoon to collect measured samples from the collection device. The patient is then required to use the tongue depressors to distribute these smaller samples into a variety of containers, jars, tubes and other collection receptacles provided to collect, store and transport the samples. Some of the tubes are bio-assay tubes with liquid (buffer solution or formalin) dedicated for diagnostic technologies or methods such as PCR (polymerase chain reaction) systems, stool cultures, PCR multiplexing systems, bio-assays and next generation genome/DNA sequencing technologies used to identify genetic bio-markers for specific pathogens in stool.

After distributing the measured samples into the various collection jars and tubes, the patient may be required to refrigerate and/or freeze the samples to limit contamination and maintain viability of the samples. The significant handling required to distribute smaller sample quantities along with the sight and odor of the stool contribute to an undignified patient experience. Patient compliance (adherence to sample submission) suffers as a result of this unpleasant, messy process. A patient's reticence to complete the stool sample collection and handling process significantly reduces sample submission to labs for testing. The resultant absence of diagnostic results can hinder a clinician's ability to diagnose and treat their patient.

There are a wide variety of stool collection, containment and transport devices and methods. Current stool collection devices lack the complete feature set required for an integrated stool sample collection, containment, distribution and transport device.

These collection devices are used for at least one or a combination of the following:
1. Stool sample collection
2. Stool sample storage
3. Patient transport of stool sample(s) to diagnostic labs
4. Stool sample containment and shipping via FedEx, UPS, USPS et. al to designated testing facilities.

There are innovations designed solely for sample collection. These simple collection devices include paper or flexible plastic sheets that are draped over a toilet lid to “catch” the sample. Another common collection method is a plastic collection bowl that sits on top of the toilet bowl (a.k.a Toilet Hat). Both of these methods expose the sample and patient to contaminants and require the patient to handle their feces with a stick or a spoon to distribute measured samples into various other collections jars.

Alternatively, there are a variety of stool sample collection devices that enable a patient to collect and store their stool sample generally for the purpose of transport or shipping whole specimens. Many of these devices employ toilet brackets or frames that are secured under the toilet seat and situated over the toilet bowl. Most of these brackets are secured under the toilet seat lid using adhesive strips, non-slip surfaces and adjustable clamps.

The majority of these collection and transport containers come equipped with a threaded cap that is fastened to the container after collection in order to prepare it for transport to the lab or shipping. These in-toilet collection devices still require a patient to defecate into a single jar or rigid container designed only for transport or storage. If the patient is required to distribute measured samples from these collection devices, the patient must manually scoop smaller samples (again, typically using wooden tongue depressors) into additional tubes and containers. The containers may also be designed for shipping samples directly to a lab or diagnostic facility via USPS, UPS or other major carriers. Many of these collection containers are dedicated to specific tests that employ next gen gene sequencing to detect the presence of certain pathogens or colon cancer. For example ColoGuard, which tests for colon cancer biomarkers, uses a toilet bracket and a rigid collection cup that is packaged and shipped directly to a specific testing facility. The aforementioned stool collection, storage and shipping containers use very similar features, function and design.

These various devices are sufficient to collect, contain and transport stool samples, However, they do not substantially or successfully mitigate some of the common factors (such as handling and dispensing) that patients find so unpleasant when submitting a stool sample for diagnostic purposes. While various stool collection methods partially reduce the unpleasant nature of stool sample collection and transport, they are not specifically designed to mitigate direct handling and do not have specific features that allow patients or lab technicians to dispense measured samples without direct handling. Without a dedicated disbursement element or a feature to distribute sample quantities from the full sample, patient compliance will continue to present a challenge to clinicians and diagnostic centers. Alternatively, an easy method is dispensing measured samples which may also be a safer and more expedient process for lab technicians and clinicians who may need to dispense smaller sample quantities themselves.

There have also been a variety of attempted approaches by others for processing stool samples into a “homogenized” state for Fecal Microbiota Transplantation (FMT). Devices for stool homogenization are not indicated for clinical diagnostic testing on patients. These approaches by others are designed to attempt to process stool collected from healthy “donors” for transplantation into “sick” patients. Devices designed for FMT homogenization are designed to collect, mix and introduce solvents into the stool sample in order to prepare the stool FMT. They use a variety of mechanical mixing embodiments, filters and ports specifically designed to isolate micro flora from the GI tract and prepare it for implantation into a patient's gut. While some stool homogenization devices integrate embodiments such as toilet brackets/frames and collection cups, they are not designed for patient use in clinical diagnostics.

No current inventions provide patients with a fully integrated system for collection, containment and dispensing (alternatively: “disbursement”) of measured (alternatively: “a portion of the whole sample”) stool samples for the purposes clinical diagnostic applications.

Accordingly, a long-felt and yet unmet need exists to change this dynamic for stool samples provided by patients for diagnostic testing. There is a need for a methodology that facilitates easier stool collection by a patient, protects the sample from bio-contamination, reduces odor and enables easier sample disbursement into collection receptacles by a patient or lab technician. This methodology can play a critical role in improving patient compliance and making diagnostic data more readily available for patients, diagnostic centers, clinicians and labs.

### SUMMARY OF PREFERRED EMBODIMENTS OF THE INVENTION

According to one preferred embodiment, the invention provides a novel device that comprises an integrated stool collection, containment, transport and sample disbursement system. The system is intended for use in the identification of disease states, infections or other physiological and psychological conditions that can be diagnosed either solely or in part by the examination of human stool.

The invention preferably includes a variety of features designed to facilitate easier patient stool sample collection, storage, and protection of the sample from bio-contamination. Other embodiments of the device reduce patient exposure to the sight, odor and potentially hazardous pathogens of stool samples. Additional features enable the disbursement of measured sample quantities into collection containers, test tubes, bio-assay tubes and other receptacles used for the collection, storage and transport of stool samples for diagnostic testing and evaluation.

According to a preferred embodiment of the invention, the stool sample collection and disbursement device is comprised of a toilet seat frame comprised of a polymeric or a heavy paper material that rests on the top of a toilet seat with one or more extensions or arms that secure the frame in place for proper patient positioning and collection. Typically rubber grommets or adhesive materials are placed on the underside of the toilet frame to maintain proper positioning and adherence to the toilet seat. The toilet frame is designed to fit towards the rear of the toilet seat for proper patient positioning and to divert urine from the collection bag. The toilet seat frame includes an opening of sufficient size and shape to accommodate the passage of patient stool samples into a collection bag that is connected to the underside of the toilet frame and within the toilet bowl. The collection bag is designed to hang above the water line of most toilets to avoid exposure to water and urine within the toilet bowl. The average waterline in most toilets is approximately 7 inches below the toilet seat.

The Collection Bag

According to a preferred embodiment of the invention, a collection bag is designed to collect, store, protect and distribute specific amounts of stool from the full sample contained within. It is typically constructed of a flexible polymeric material such as EVA or PVC or other flexible materials that are designed to contain stool without contaminating the sample, altering the microflora of the stool or sample. The collection bag may also be comprised of other materials. For example, the collection bag may be constructed of paper or other biodegradable materials to provide a more environmentally friendly alternative to plastic by reducing its carbon footprint. The polymeric material of the collection bag may be textured or colored to reduce visual exposure to the sample. The collection bag may comprise an area for labels that designate patient information and tests performed on the specimen.

Odor reduction during collection or disbursement may further improve the collection bag using a variety of systems or features. This can be accomplished during collection using scented material incorporated into the bag material. The bag may also include a valve with a filtration component (for example carbon or charcoal activated). The valve can be incorporated into the bag itself, the nozzle or both. The valve should be bi-directional in order to prevent leaking.

Additional aspects of the invention may include certain components that allow the bag to stand upright and avoid spillage prior to sealing the bag. For example, an arm connected to the side of the bag may extend to create a brace, which would work similarly to a kickstand on a bicycle. The collection bag can have a flat or gusseted bottom to help keep the bag upright on a flat surface without tipping over and spilling the contents of the collection bag. Alternative shapes and sizes or thickness of the bag material may also be used to keep the bag positioned upright to reduce spilling.

The Collection Bag Connection to the Frame

According to a preferred embodiment of the invention, the collection bag is secured to the underside of the toilet frame directly under the opening of the toilet frame. This embodiment allows the collection bag to suspend or hang within a toilet bowl and above the waterline inside of a toilet bowl. The opening of the collection bag should be of sufficient size and shape to allow passage of the stool sample through the opening in the toilet frame and into the collection bag to prevent spilling or leaking.

Typically the collection bag is secured to the underside of the toilet frame using flexible tabs. The tabs are secured to the sides of the toilet frame using adhesive or integrated hooks on the toilet frame. The tabs extend from the toilet frame into the inside walls of the collection bag. The opposite ends of the tabs are secured to the inside of the bag using either heat sealing or adhesive.

This embodiment creates a funnel that allows the stool sample to pass through the opening of the toilet frame and into opening of the collection bag.

In alternate embodiments, the collection bag and toilet frame can be secured using a direct connection between the toilet frame and collection bag with adhesive, heat sealing or by using clips of a variety of shapes and sizes to accomplish this connection.

The connection between the collection bag will be of sufficient strength to accommodate a sample without separating from the toilet frame and falling into the toilet bowl below.

In alternate embodiments, the toilet frame may not be a necessary component of the device. The collection bag may connect directly to the toilet seat using flaps, adhesive tabs, suction cups, elastic bands or other means of connecting the collection bag securely to the toilet bowl and/or the toilet bowl lid.

Separating the Collection Bag from the Toilet Frame

The tabs that connect the collection bag and toilet frame are typically perforated. These perforations provide easier separation of the frame and the collection bag after the sample has been collected.

Typically a pull string with a looped handle connected to the tabs can be used for separation. The string is attached to the tabs or the connection between the bag and the toilet frame. Pulling the looped handle provides sufficient tension to shear the tabs along the perforations and complete the separation.

This separation can be also accomplished using a variety of structures, methods or materials such as additional polymeric or paper loops, handles or string connected to the tabs on the collection bag,

In alternate embodiments the collection bag itself can be pulled in order to complete the separation and without the need for a handle. For example, the collection bag may include tabs, loops or other embodiments such as a texture or ribbing on the surface of the bag material or openings within the bag that enable one to separate the collection bag from the toilet frame.

Sealing the Collection Bag after Stool Collection

After separation from the toilet frame, the collection bag is closed and sealed to prevent leakage of the contents from inside the bag and protect the specimen during patient handling, disbursement, transport and storage.

The collection bag is typically sealed primarily by a zipper lock closure or a plastic slider closure. The seal creates a primary barrier to prevent contents inside the bag from escaping. Other methods of sealing the bag may be adhesive strips, bonding elements, clips or fasteners.

According to other preferred embodiments, the invention may also include a secondary seal designed to reinforce the primary seal and create an airtight, hermetically sealed environment to protect the sample and withstand the external pressure necessary to distribute samples.

The secondary seal may use a flap secured to one side of the collection bag which folds over the top of the bag and adheres to the opposite side. This seal can also be accomplished using other adhesive materials such as Velcro or pressure sensitive adhesive.

In alternate embodiments, the secondary closure may not be necessary provided that the primary closure is strong enough to create an airtight seal and withstand the external pressure required to store, transport and distribute samples.

The Dispensing Port and Nozzle

According to preferred embodiments of the invention, the collection bag contains a method of sample disbursement to allow patients to distribute measured amounts of stool into the various collection tubes and containers. Typically the embodiment includes a dispensing port integrated into the bag similar to those used in IV bags or other clinical infusion bags. For instance a welded port using RF (Radio Frequency) welding or similar technology for manufacturing ports or tubing in bags designated for clinical use. The dispensing port should be of sufficient diameter to allow stool of any consistency to flow freely out of the bag. For instance, a port with inner dimensions of no less than about 0.05″ and no greater than about 5.″

In alternative embodiments, a length of polymeric tubing can extend from the dispensing port. The extended tubing is typically comprised of a polymeric or surgical tubing material designed for I.V. infusions and clinical applications. The tubing can be flexible or rigid with a length of no shorter than about 0.05″ and no longer than 10″. The tubing may be used to distribute sample quantities without a nozzle. The end of the tubing can be sealed and resealed using luer locks, clamps or caps of sufficient strength to avoid leaking or unintended opening.

Typically, a re-sealable nozzle or valve is affixed to the port on the collection bag. The nozzle provides a means to distribute measured samples in a controlled manner and can be re-sealed after sample disbursement is completed. The nozzle is secured directly to the dispensing port using heat welding, a threaded connection or adhesives such as glue or tape.

In alternative embodiments, the nozzle can be attached to the extended tubing from the dispensing port (as described above), which may provide a more expedient method to distribute samples through the nozzle at various angles.

The nozzle is typically equipped with an attached cap that allows the bag to be re-sealed after measured samples have been distributed. The cap or closure system should be sufficiently rugged and secure to prevent leaking or inadvertent spillage when it is closed.

The nozzle or port may also be opened and sealed using a variable flow valve. In this embodiment, the flow rate could be increased, decreased or sealed by twisting the nozzle. This nozzle design could employ an internal plunger mechanism within the nozzle that is raised or lowered by twisting the nozzle. A variety of shapes and sizes within the inner diameter of the nozzle can be employed to provide the most expedient and accurate sample disbursement.

Typically, the desired sample quantity may be extracted by opening the valve, nozzle or both and applying positive pressure to the outside of the bag. Applying positive pressure (squeezing) to the outside of the collection bag pushes the specimen through the nozzle, port or both to create a specimen flow typically analogous to employing an icing dispenser used in baking. The force required to distribute a sample through the nozzle is dependent on the consistency of the stool sample within the collection bag and the diameter of the port and/or inner diameter of the nozzle.

Additional Aspects of the Invention

Aspects of this invention also provide a variety of devices or methods in the invention that can use a patient's collected stool sample to further diagnose or detect the presence of infectious gastrointestinal pathogens, inflammatory bowel disease (IBD, Crohn's Disease, Colitis) and other disease states of the gut and the human body including psychiatric and behavioral health disease.

There are aspects of the invention that also provide systems and methods in which the collection bag may further be used to contain buffer solution, formalin fixative, formaldehyde or other liquid stool transport mediums. These solutions help preserve the sample and enable expedient transfer of sample material into various testing platforms such as stool cultures, PCR (Polymerase Chain Reaction), Multiplex PCR, bio-assays, blots, genetic biomarker tests (DNA micro arrays), other genomic or sequencing techniques and/or similar technologies either currently or subsequently developed in the art to determine the presence, absence or acuity of digestive disease(s) or pathologies with patient stool samples.

There are aspects of the invention that also provide systems and methods that can detect, identify or diagnose stool-borne illnesses of patients in the home.

After collection of the stool sample by the invention, these embodiments can provide patients with more rapid and convenient results. For instance, the collection bag may include sensors using PCR, PCR Multiplex, bio-assay, DNA micro-array, other genomic sequencing technologies and/or similar technologies either currently or subsequently developed in the art to determine the presence, absence or acuity of digestive disease(s) or infectious pathogens such as C-Difficile and intestinal parasites, diarrhea caused by travelers diseases and other gastrointestinal disease states that can be diagnosed with the patient's stool sample. The sensors may be integrated into the material of the collection bag or perhaps added to the bag using adhesive or other means of integration within the collection bag or collection system depending on the symptoms of the patient.

The collection bag may also include current rapid tests that detect human hemoglobin in fecal samples; also known as fecal occult blood tests (FOBT). There are aspects of this invention that provide systems and methods to collect urine samples from patients for diagnostic purposes.

There are aspects of the invention that also provide systems and methods to analyze the lining of the gut. For instance the epithelial cells in the lining of the intestine that may be shed with or without the stool. The pathogens that line the gut are also considered to be an additional aspect of the microbiome and can provide additional insights into the health of the microbiome. For instance certain cells within the lining of the gut can contain bacteria that may detect the presence of a pathogen such as B. Fragilis. These cells may be shed in the stool or separately and can provide insights into a patient's microbiome.

There are aspects of the invention that also provide systems and methods to collect urine from a patient for diagnostics and analysis. Urine collection also remains burdensome and the collection process may be significantly improved using an alternative embodiment of the device.

There are aspects of the invention that also provide systems and methods to collect and transport infectious diarrhea in underdeveloped global areas where diarrhea is a dire health risk and significant cause of mortality; specifically in children. Current efforts to provide more sanitary and healthy means of collecting and disposing of human feces have been less than ideal and infectious diarrhea still remains a leading global killer. An alternate embodiment of the device may provide a means to reduce the risk of infectious disease by serving as a alternative to current methods of reducing unhealthy and unsanitary methods of disposing feces and defecation.

There are aspects of the invention that also provide systems and methods that can be used my military personnel for the collection and transport of waste in the field. Alternate embodiments of the invention may help reduce current unsanitary and less covert methods used by members of the military to dispose of their waste.

### BRIEF DESCRIPTION OF THE DRAWINGS

FIG. 1 depicts a proposed solution that includes a thin polymeric toilet seat cover (a.k.a. “Toilet Frame”) with foam ring around the opening in the center and appendages to secure the toilet frame on the top of the toilet seat.

FIG. 2 depicts a top view of the toilet frame situated on a toilet seat. Flexible tabs attach to integrated hooks (aka Toilet Frame Fasteners) on the sides of the toilet frame and suspend a flexible, polymeric stool sample collection bag (not shown) above the water line in the toilet bowl.

FIG. 3 depicts a cut-away side view of the toilet seat frame situated on the toilet with the collection bag suspended via the flexible tabs and toilet frame fasteners below the toilet frame and above the water line in the toilet. A full side view of the collection bag within the bowl demonstrates the interface between the toilet frame and collection bag when the device is situated on the toilet seat. The attachment of the flexible tabs and the inside walls of the collection bag with perforated tear lines is also depicted.

FIG. 4 depicts a close view of the collection bag attached to the toilet frame with a pull string and looped handle. The pull string and looped handle enable the collection bag to detach from the toilet frame after stool collection has been completed. An additional illustration depicts the separation of toilet frame and collection bag as the looped handle is pulled away from the bag.

FIG. 5 depicts a cut-away side view of the integrated dispensing port with extended tubing and a re-sealable cap. An alternate embodiment depicts a variable flow nozzle attached directly to the dispensing port without extended tubing.

FIG. 6 depicts a side view of the collection bag with the primary seal and the secondary flap with adhesive. The secondary flap folds over the primary seal to ensure integrity of the collection bag and dispensing port due to external pressures during sample disbursement, storage and transport.

### DESCRIPTION OF PREFERRED EMBODIMENTS OF THE INVENTION

Certain preferred embodiments of the invention are now described in detail. It is to be understood that these preferred embodiments do not limit the broad scope of the invention in any manner whatsoever.

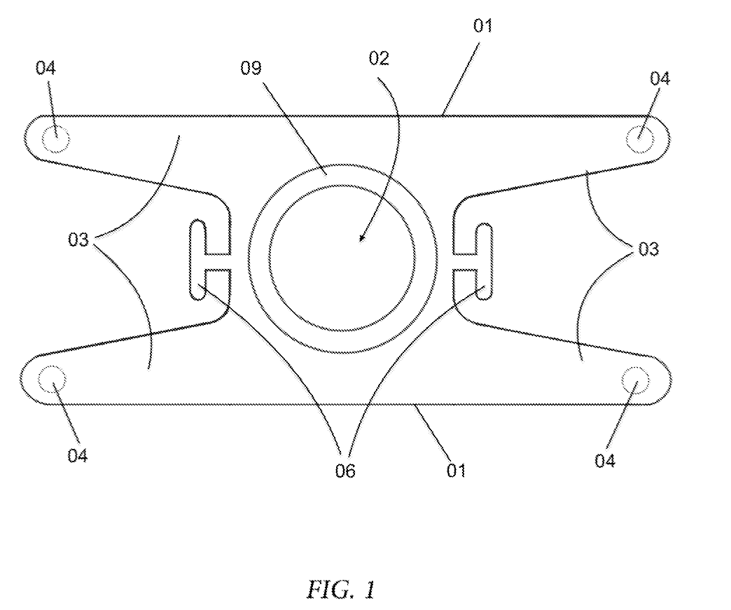

Certain preferred embodiments of the invention are illustrated in FIG. 1. FIG. 1 is a top view of the polymeric toilet frame 01 with four extended arms and an opening in the center 02. The opening 02 is designed to accommodate the collection bag 07 (not shown), which is suspended below the opening 02 and into the toilet bowl. The number of extended toilet seat arms 03 can be altered to less than four or more than four. The frame 01 can be moved forward or backward to accommodate the patient. Toilet frame fasteners 06 connect to the flexible tabs 05 (not shown) that are also attached to the inner walls of the collection bag. When the flexible tabs are connected to the toilet frame fasteners 06, this connection widens the opening of the collection bag 07 underneath the toilet frame 01 to create a funnel for which fecal samples from the patient are deposited without spilling or mess. A foam ring 09 surrounds the edge of the opening 02 and is designed for patient comfort and proper positioning. The foam ring 09 can be made of open cell foam, rubber or other similar types of soft material for this purpose. The toilet frame 01 can be constructed from other materials such as paper or recyclable materials in order to reduce the carbon footprint of the device.

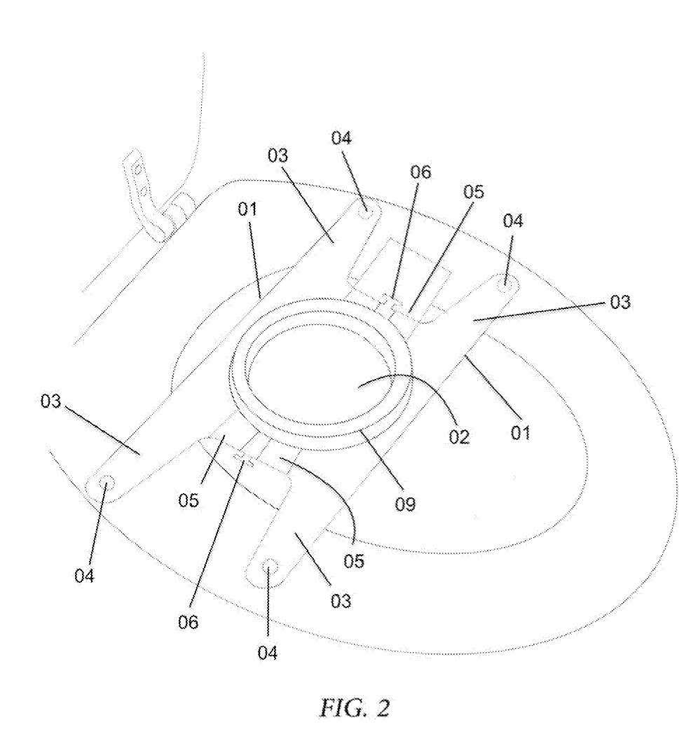

Certain preferred embodiments of the invention are illustrated in FIG. 2. FIG. 2 is a view of the toilet frame 01 positioned on a toilet seat. The foam ring 09, which is intended for patient comfort and proper positioning, is demonstrated surrounding the opening 02. The flexible tabs 05 are looped over the toilet frame fasteners 06. The flexible tabs 05 are made of thin plastic sheeting and “looped” over the toilet frame fasteners 06 in order to create a funnel into the opening of the collection bag (not shown) underneath. Rubber grommets 04 are adhered to the underside of the toilet frame in order to prevent slipping or shifting while the patient is positioned on the toilet frame 01.

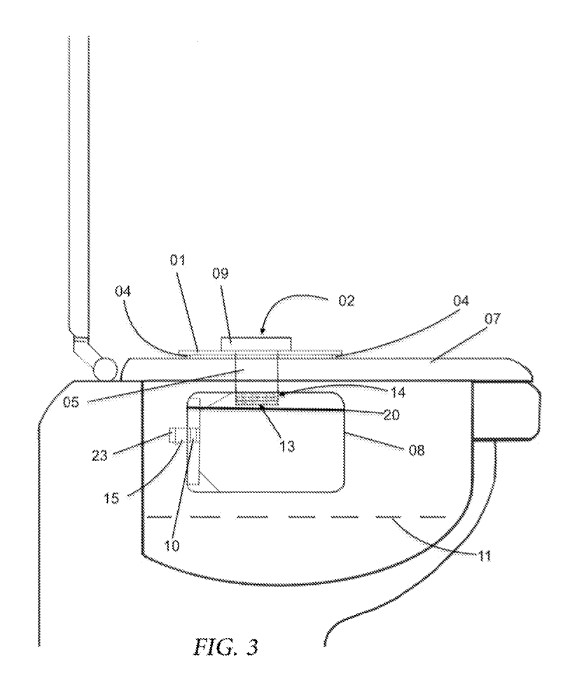

Certain preferred embodiments of the invention are illustrated in FIG. 3. FIG. 3. is a cut-away, side view of a toilet bowl with the device in place on the toilet seat. The toilet frame 01 and collection bag 08 are connected by the two flexible tabs 05 which position the collection bag 08 beneath the toilet frame and above the water line of the toilet 11. The side view of the frame 01 demonstrates the foam ring 09 over the opening of the toilet frame 01 along with the rubber grommets 04 beneath the toilet frame arms 03. The collection bag 08 has a RF welded port similar to those produced within IV bags and other medical infusion bags. The dispensing port 10 is comprised of a short section of extended tubing and a sealable cap. The collection bag is suspended from toilet frame fasteners 06 (not shown) on the toilet frame 01 that connect flexible tabs 05 to the inside walls of the collection bag and suspend the collection bag 08 beneath the toilet frame 01. The collection bag is separated from the toilet frame by a pull string (not shown) with a looped handle (not shown) which help tear the flexible tabs 05 from the inside walls of the collection bag 08. The separation is expedited by perforations 14 along the flexible tabs 05 inside the collection bag 08 which allow them to tear with minimal effort.

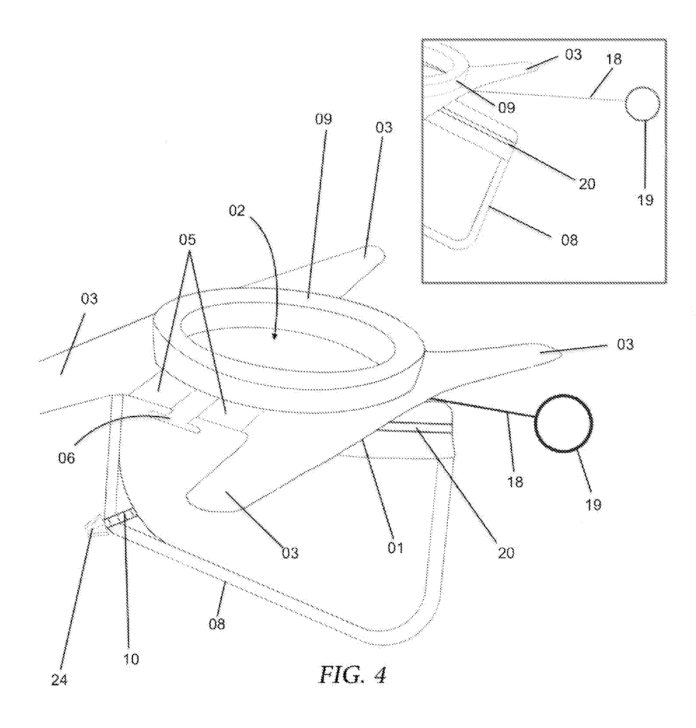

Certain preferred embodiments of the invention are illustrated in FIG. 4. FIG. 4 is a side illustration depicting the separation of the collection bag 08 from the toilet frame 01 after a stool sample has been deposited by the patient into the collection bag 08. A string with a pull ring 18 is attached to the inside of the collection bag 08. When the pull string 18 with the pull handle 19 is pulled, the string tears the flexible tab connection 13 inside the collection bag 08 and separating the toilet frame from the collection bag 08. A smaller illustration depicts the toilet frame 01 and collection bag 08 separating as the pull string 18 and looped handle 19 are pulled.

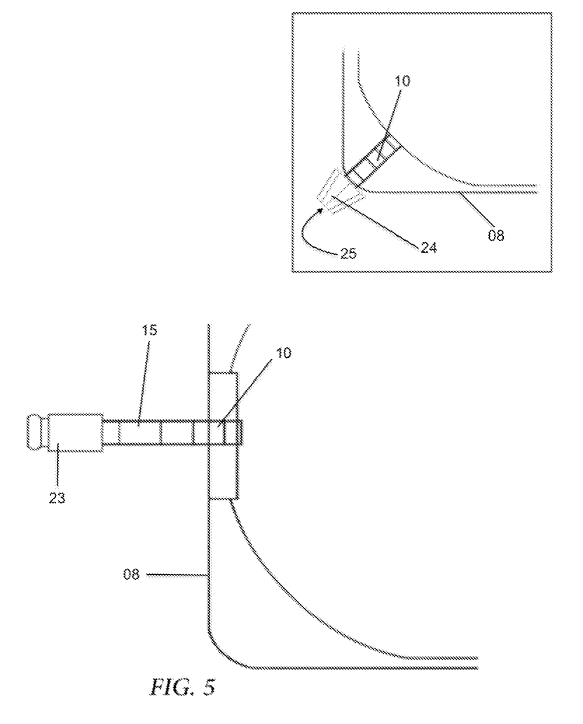

Certain preferred embodiments of the invention are illustrated in FIG. 5. FIG. 5 is a close up, cut-away illustration of the dispensing port 10 in the collection bag 08. The dispensing port 10 is the means by which external pressure to the outside of the collection bag 08 enables disbursement of stool from the collection bag. The ports are typically RF welded as similar to ports in IV bags or other clinical bags with ports for infusion or that utilize ports for fluids. A section of extruded surgical tubing 15 is connected to the port 10 and extends from the port 10 to a removable cap 23. The extended tubing 15 can vary in length from 0.01″ to 10″ from the opening of the port 10 in the collection bag 08. In alternate embodiments, the ports can be produced using other means such as creating an opening or a hole in the collection bag. The extended tubing 15 from the port 10 includes a re-sealable cap 23 on the end of the tubing 15 to prevent leaking from the collection bag 08. The re-sealable collection bag 08 allows sample disbursement and subsequent storage, transport or further disbursements as necessary.

Also demonstrated is a small illustration of an alternate embodiment that connects a variable flow nozzle 24 directly to the port. This variable flow nozzle 24 enables adjustment of the dispensing rate depending on the consistency of the stool sample within the collection bag. In alternate embodiments, the variable flow nozzle 24 can include a cap 23 at the end or use a mechanism such as an internal plunger mechanism to close the nozzle.

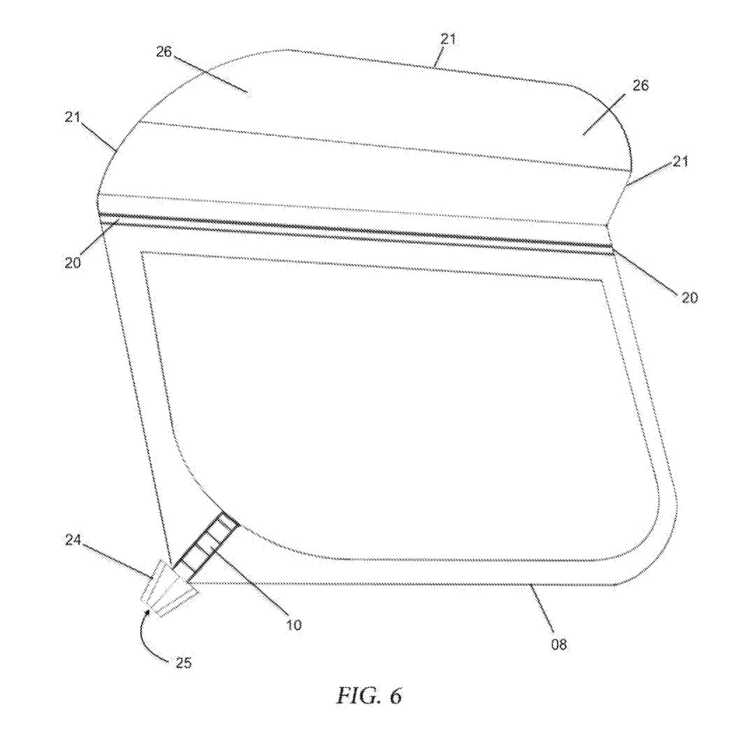

Certain preferred embodiments of the invention are illustrated in FIG. 6. FIG. 6 is a side view of the collection bag 08 after it has been detached from the toilet frame 01 and sealed using the primary zip lock seal 20. The collection bag has a volume of at least 1 mL and at most 1500 mL. The primary seal 20 is shown using a zip-lock type closure system. In alternate embodiments, the closure system can be a slider zipper seal, adhesive or another means by which the collection bag 08 can be closed in order to prevent leaking or spilling. Based on an alternate embodiment the dispensing port 10 is connected directly to a variable flow nozzle 24 and an internal closure mechanism 25 within the variable flow rate nozzle. The secondary flap 21 is attached to the side of the collection bag 08 (not shown). An adhesive strip is shown at the top of the flap. After the primary seal 20 has been closed, the secondary flap 21 folds over the top of the primary seal and adheres to the opposite side of the collection bag 08. The secondary seal 21 provides additional protection of the sample and prevents leaking or contamination of the sample. In alternate embodiments, a secondary flap 21 may not be required if current or subsequent technologies in the art can produce a primary flap 20 that is of sufficient strength to withstand the external pressure applied to the outside of the collection bag during disbursement or other factors that may cause the primary seal to inadvertently open. The collection bag 08 is designed to withstand external pressures of at least 0.025 PSI (pounds per square inch) up to 1000 PSI. The external pressure required to dispense stool from the bag 08 varies based on the consistency and flow rate of the stool sample in the bag.

1. A method of collecting, containing and dispensing stool samples from a patient for diagnostic testing or clinical analysis, the method comprising: a toilet frame with an opening in the center rests on a toilet seat over the opening of the toilet; providing a flexible, polymeric collection bag which is affixed to the underside of the opening of the toilet frame; collecting a stool sample from a patient as the patient is seated on the toilet frame and defecating into the collection bag; the collection bag is separated from the toilet frame after collection of the patient's stool sample; the collection bag is sealed to protect the sample and ensure the integrity of the collection bag; external positive pressure is applied to the outside of the bag to dispense measured amounts of stool; the collection bag having a re-sealable port, wherein a quantity of the patient's stool is dispensed from the collection bag for diagnostic analysis and testing.

2. The method of claim 1, wherein an opening in the center of the toilet frame is mated to the opening of the collection bag within the toilet bowl creating a funnel which allows the stool sample to flow into the collection bag as the patient is defecating.

3. The method of claim 1, wherein the funnel connecting the toilet frame to the collection bag protects the sample from urine and toilet water.

4. The method of claim 1, wherein the toilet frame is affixed to the collection bag by thin flexible tabs or adhesive.

5. The method of claim 1, a foam or rubber ring surrounding the opening of the toilet frame for patient comfort and ensure proper positioning while seated on the toilet frame.

6. The method of claim 1, wherein the collection bag is designed to hold, transport and dispense measured amounts from only one stool sample collected by a patient.

7. The method of claim 1, wherein the collection bag has a volume of at least 1 mL and at most 1500 mL.

8. The method of claim 1, wherein the flexible flaps are perforated to separate the collection bag from the frame.

9. The method of claim 1, wherein the collection bag has a dispensing port (a.k.a. “port”).

10. The method of claim 1, wherein the port is comprised of an RF welded port similar to those integrated into I.V. bags or medical bags used for infusion.

11. The method of claim 8, wherein the port is for dispensing stool from a patient only.

12. The method of claim 1, wherein the port has extruded tubing ranging from approximately 0.01″ to 10″ from the opening of the port.

13. The method of claim 1, wherein the dispensing port has an inner diameter width of at least 0.02″ and at most 4″.

14. The method of claim 1, wherein the dispensing port has an outer diameter of at least 0.03″ and at most 4.5″.

15. The method of claim 1, wherein the collection bag dispensing port is re-sealable.

16. The method of claim 10, wherein the port has a removable cap, clamp or luer locking mechanism.

17. The method of claim 17, wherein the cap on the port can be a variable flow nozzle.

18. The method of claim 18, wherein the nozzle is re-sealable using a cap or integrated closure mechanism.

19. The method of claim 1, wherein dispensing smaller or measured amounts of stool is accomplished by applying external pressure to the outside of the collection bag.

20. The method of claim 20, wherein applying external pressure (i.e. “squeezing”) to the collection bag precipitates the flow of stool through the port or nozzle.

21. The method of claim 1, wherein the collection bag can withstand external pressure in the range of 0.025 pounds per square inch (PSI) and 1000 pounds per square inch (PSI).

22. The method of claim 1, wherein the collection bag seal is comprised of a zip lock or zipper slider closure or other means of sealing the bag including clips, adhesive, velcro.

23. The method of claim 22, wherein the collection bag comprises a secondary adhesive flap that folds over the top of the primary seal to ensure the integrity of the bag while dispensing stool through the dispensing port.

24. The method of claim 6, wherein the collection bag contains a buffer solution, formalin fixatives or formaldehyde to preserve a single stool sample during transport.

25. The method of claim 6, wherein the collection bag comprises sensors or material by which the patient's collected stool sample in-situ can identify pathogens, infectious material, parasites, genomic biomarkers, and GI micro-flora for diagnostic purposes.

26. The method of claim 6, wherein the sensors comprise testing platforms including Polymerase Chain Reaction (PCR), Multiplex PCR, bio-assays, blots, genetic biomarker tests other genomic tests or similar technologies either currently in practice or subsequently developed in the art to determine the presence, absence or acuity of digestive diseases or pathologies within a patient's stool sample.
